(19) United States
(12) Patent Application Publication
(10) Pub. No.: US 2024/0249842 A1
(43) Pub. Date: Jul. 25, 2024

(54) INFORMATION PROCESSING DEVICE

(71) Applicant: NEC Corporation, Tokyo (JP)

(72) Inventor: Yuki KOSAKA, Tokyo (JP)

(73) Assignee: NEC Corporation, Tokyo (JP)

(21) Appl. No.: 18/420,890

(22) Filed: Jan. 24, 2024

Related U.S. Application Data
(63) Continuation of application No. 18/567,875, filed on Jan. 1, 0001.

(30) Foreign Application Priority Data
Jan. 23, 2023 (WO) PCT/JP2023/001970

(51) Int. Cl. G16H 50/20

(52) U.S. Cl. CPC G16H 50/20

(57) ABSTRACT

An information processing device 100 of the present disclosure includes: an acquisition unit 121 that acquires a model that is generated for each elapsed period, and has learned by machine learning to output a measure for a human by receiving input of a plurality of types of feature value representing a condition of the human; a collection unit 122 that collects first output that is obtained when a predetermined number of types of feature value are input to the model of each elapsed period, and second output that is obtained when some types of feature value in the predetermined number of types of feature value are input to the model of each elapsed period; and a setting unit 123 that sets, on the basis of the first output and the second output, types to be associated with the model of each elapsed period. Thereby, the information processing device 100 can be used for assistance of decision-making by a user, or the like.

This application is a Continuation of U.S. application Ser. No. 18/567,875, filed Dec. 7, 2023, which is a National Stage Entry of PCT/JP2023/032508 filed on Sep. 6, 2023, which claims priority from PCT International Application PCT/JP2023/001970 filed on Jan. 23, 2023, the contents of all of which are incorporated herein by reference, in their entirety.

### TECHNICAL FIELD

The present disclosure relates to an information processing device, an information processing method, and a program.

### BACKGROUND ART

There is growing demand for personalized medicine in which treatment methods are decided depending on the conditions of individual patients when those patients are treated. Personalized medicine aims to provide treatment methods appropriate for the condition of each patient by using genetic information, medical care information, and the like of individuals. Here, PATENT LITERATURE 1 describes selection of treatment methods by using a computer.

### CITATION LIST

### Patent Literature

PATENT LITERATURE 1: Japanese Unexamined Patent Application Publication (Translation of PCT Application) No. 2016-514291

### SUMMARY OF INVENTION

### Technical Problem

However, as described above, when a treatment method is to be selected, it is necessary to collect various feature values such as bio-information representing the condition of a patient. Since collection of information about patients requires costs and time, for prompt and easy selection of an appropriate treatment method, it is necessary to enhance the efficiency of collection of information about patients. Then, such a problem occurs not only in treatment, but occurs also in the case where methods for training, exercise, dieting or the like are provided, and it is necessary to enhance the efficiency of collection of information about subjects for measure proposals.

Therefore, an object of the present disclosure is to provide an information processing device that can enhance the efficiency of collection of information about subjects for measure proposals.

### Solution to Problem

An information processing device according to one aspect of the present disclosure is configured to include:
an acquisition unit that acquires a model that is generated for each elapsed period, and has learned to output a measure for a human by receiving input of a plurality of types of feature value representing a condition of the human;
a collection unit that collects first output that is obtained when a predetermined number of types of feature value are input to the model of each elapsed period, and second output that is obtained when some types of feature value in the predetermined number of types of feature value are input to the model of each elapsed period; and
a setting unit that sets, on the basis of the first output and the second output, types to be associated with the model of each elapsed period.

Further, an information processing method according to one aspect of the present disclosure is configured to include:
acquiring a model that is generated for each elapsed period, and has learned to output a measure for a human by receiving input of a plurality of types of feature value representing a condition of the human;
collecting first output that is obtained when a predetermined number of types of feature value are input to the model of each elapsed period, and second output that is obtained when some types of feature value in the predetermined number of types of feature value are input to the model of each elapsed period; and
on the basis of the first output and the second output, setting types to be associated with the model of each elapsed period.

A program according to one aspect of the present disclosure is configured to cause a computer to execute processing to:
acquire a model that is generated for each elapsed period, and has learned to output a measure for a human by receiving input of a plurality of types of feature value representing a condition of the human;
collect first output that is obtained when a predetermined number of types of feature value are input to the model of each elapsed period, and second output that is obtained when some types of feature value in the predetermined number of types of feature value are input to the model of each elapsed period; and
on the basis of the first output and the second output, set types to be associated with the model of each elapsed period.

### Advantageous Effects of Invention

By being configured in the manner above, the present invention can enhance the efficiency of collection of information about subjects for measure proposals.

### BRIEF DESCRIPTION OF DRAWINGS

FIG. 1 is a block diagram illustrating a configuration of an information processing device according to a first exemplary embodiment of the present disclosure.

FIG. 2 illustrates a state of data processing by the information processing device disclosed in FIG. 1.

FIG. 3 illustrates a state of the data processing by the information processing device disclosed in FIG. 1.

FIG. 4 illustrates a state of the data processing by the information processing device disclosed in FIG. 1.

FIG. 5 illustrates a state of the data processing by the information processing device disclosed in FIG. 1.

FIG. 6 illustrates a state of the data processing by the information processing device disclosed in FIG. 1.

FIG. 7 illustrates a state of the data processing by the information processing device disclosed in FIG. 1.

FIG. 8 is a flowchart illustrating an operation of the information processing device disclosed in FIG. 1.

FIG. 9 is a block diagram illustrating a hardware configuration of an information processing device according to a second exemplary embodiment of the present disclosure.

FIG. 10 is a block diagram illustrating a configuration of the information processing device according to the second exemplary embodiment of the present disclosure.

### DESCRIPTION OF EMBODIMENTS

### First Exemplary Embodiment

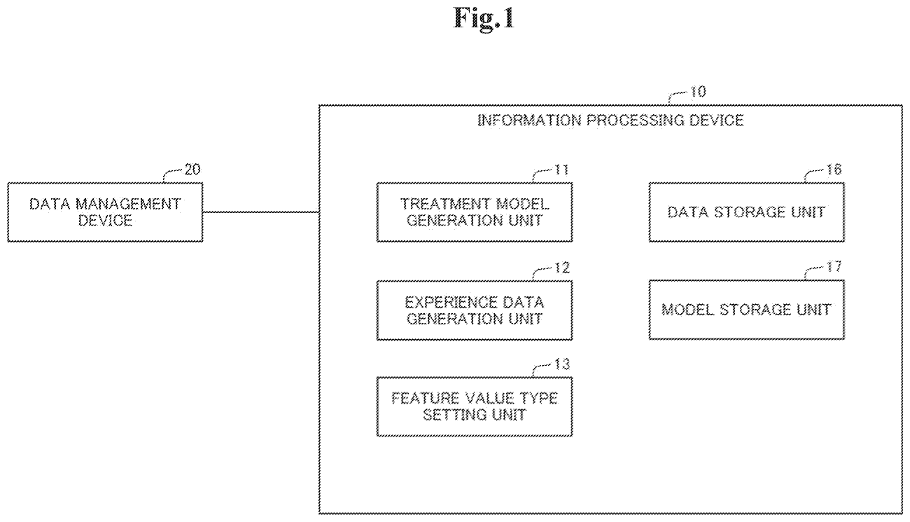

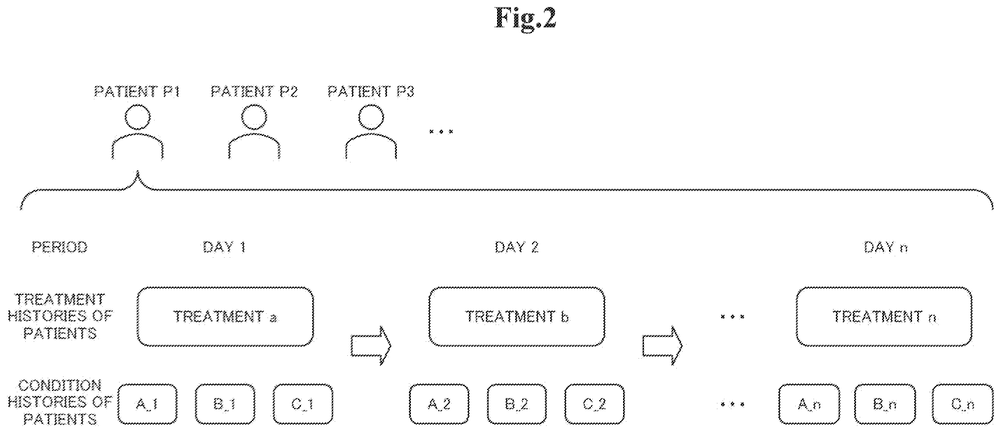

A first exemplary embodiment of the present invention will be described with reference to FIGS. 1 to 8. FIG. 1 is a diagram for explaining a configuration of an information processing device, and FIGS. 2 to 8 are illustrations for explaining the processing operation of the information processing device.

### [Configuration]

An information processing device 10 according to the present disclosure is used for proposing treatment methods according to the conditions of individual patients when the patients are treated. In particular, the information processing device 10 has the function of specifying necessary types of patient information representing the conditions of patients for treatment decision models, each of which is generated corresponding to an elapsed period during treatment, and outputs a treatment method. Note that the information processing device 10 of the present invention may be used also for providing measures (e.g. treatments, menus, behaviors, proposals) that can be implemented in accordance with the conditions of subject humans not only in treatment, but also in training, exercise, dieting, and the like.

The information processing device 10 includes one or more information processing devices each including an arithmetic unit and a storage device. Then, as illustrated in FIG. 1, the information processing device 10 includes a treatment model generation unit 11, an experience data generation unit 12, and a feature value type setting unit 13 that are constructed by execution of a program by an arithmetic unit. Further, the information processing device 10 includes a data storage unit 16 and a model storage unit 17 that are formed in a storage device. Hereinafter, each configuration will be described in detail.

The treatment model generation unit 11 (acquisition unit) generates treatment decision models (models) that output treatment methods (measures) according to the conditions of patients (humans). Specifically, the treatment model generation unit 11 first acquires, as learning data for machine learning, patient information from a data management device 20, receives input of the patient information, and stores the patient information on the data storage unit 16. For example, the patient information as the learning data includes a condition history and a treatment history of a patient for each elapsed period. As an example, FIG. 2 illustrates a conceptual diagram of the patient information. In this example, patient information about a patient P1 first includes “dates” that are set as elapsed periods from the start of treatment, and each stage which is an elapsed period is set as “Day 1”, “Day 2”, . . . . Then, the patient information includes a condition history of the patient and a treatment history of the patient for each stage, and includes a condition history “A_1”, “B_1”, and “C_1” of the patient, and a treatment history “Treatment a” of the patient at the stage “Day 1”, a condition history “A_2”, “B_2”, and “C_2” of the patient, and a treatment history “Treatment b” of the patient at the stage “Day 2”. It is assumed here that each condition history of a patient includes a plurality of types of feature value representing the condition of the patient, the types of feature value include, for example, information such as the age, height, body weight or medical history of the patient, bio-information obtained by measurement such as the body temperature, blood pressure, heart rate or blood components values of the patient, and the like, and different alphabetical letters like “A_” “B_”, and “C_” represent different types in the example of FIG. 2. Further, different alphabetical letters like “Treatment a” and “Treatment b” represent different treatment methods in the example of FIG. 2. Then, the patient information includes information about each of a plurality of humans, patients P1, P2, and P3.

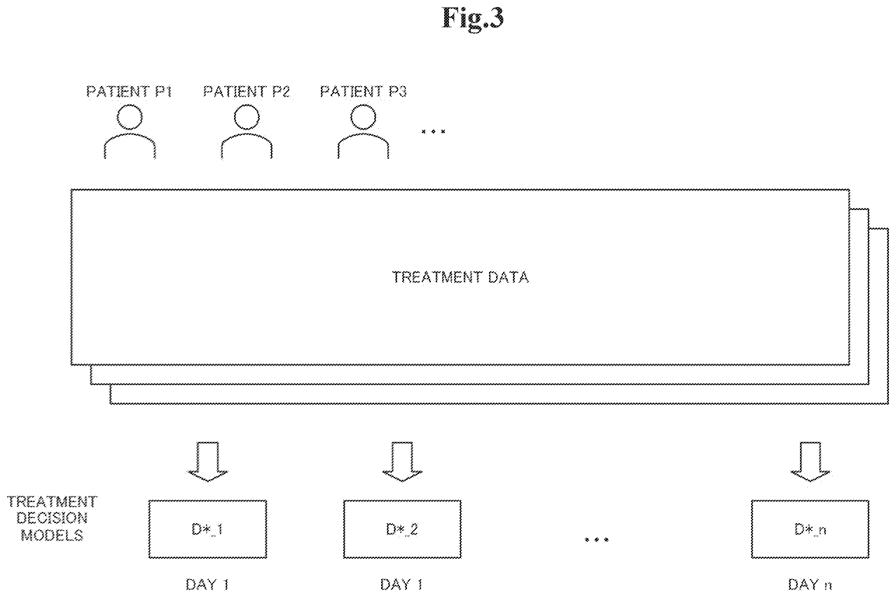

Then, as illustrated in FIG. 3, the treatment model generation unit 11 learns, for each stage which is an elapsed period, the patient information about the plurality of humans, the patients P1, P2, P3, . . . , and generates treatment decision models “D*_1”, “D*_2”, and “D*_3” for each stage. Specifically, for example, when generating a treatment decision model of the stage “Day 1”, the treatment model generation unit 11 learns input of all types of feature value “A_1”, “B_1”, and “C_1”, which are the condition history of each patient of “Day 1”, to output the treatment history “Treatment a” of each patient of “Day 1”, and generates the treatment decision model “D*_1” of “Day 1”. Further, for example, when generating a treatment decision model of the stage “Day 2”, the treatment model generation unit 11 learns input of the condition history of each patient up until “Day 2”, i.e. all types of feature value “A_1”, “B_1”, “C_1”, “A_2”, “B_2”, and “C_2”, which are the condition histories of each patient of “Day 1” and “Day 2”, to output the treatment history “Treatment b” of each patient of “Day 2”, and generates the treatment decision model “D*_2” of “Day 2”. Note that whereas only “A_”, “B_”, and “C_” are illustrated as all types (a predetermined number of types) of feature value which are condition histories input at the time of the learning in the explanation described above, it is assumed that there are actually many more types. Further, the number of types of feature value input at the time of the learning may be not only the number of all prepared types, but also a preset number (predetermined number) of types.

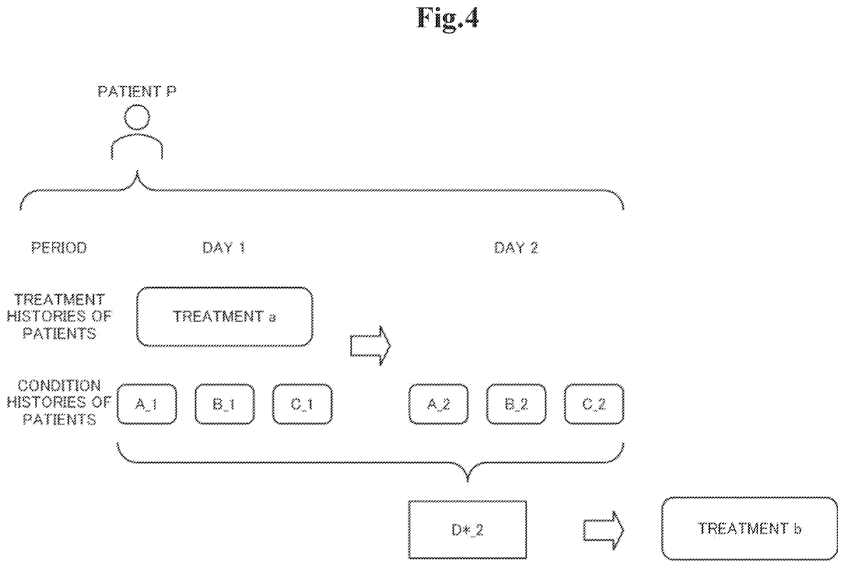

The treatment decision models generated in this manner are configured to receive input of condition histories of a patient for whom a treatment method has not been decided at a predetermined stage to thereby output a treatment method at that stage. For example, as illustrated in FIG. 4, regarding a patient P for whom a treatment method of “Day 2” has not been decided, condition histories up until “Day 2”, i.e. a plurality of types of feature value “A_1”,

“B_1”, “C_1”, “A_2”, “B_2”, and “C_2”, which are condition histories of “Day 1” and “Day 2”, are input to the treatment decision model “D*_2” of “Day 2”, which is the relevant stage, and thereby the treatment method “Treatment b” of “Day 2” is output. Note that elapsed periods relevant to stages described above may be not only the periods in units of “days”, but also any periods such as periods in units of “months”, for example.

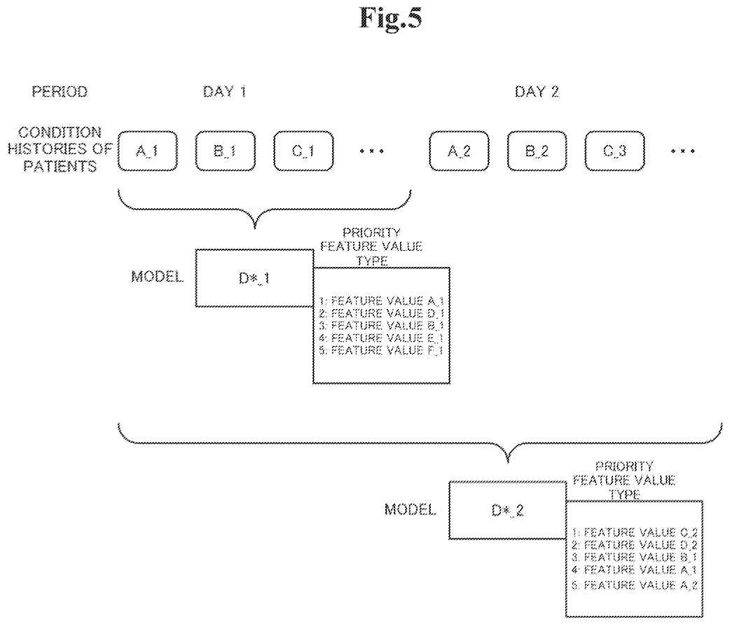

Further, the treatment model generation unit 11 prioritizes types of feature value that influence each of the treatment decision models “D*_1”, “D*_2”, and “D*_3” of a stage generated as described above. For example, since the treatment model generation unit 11 sets a weight for each type of feature value along with the execution of the machine learning of the treatment decision models described above, the treatment model generation unit 11 sets places in orders of priority of the types of feature value depending on the values of the weights. As an example, as illustrated in FIG. 5, for the treatment decision model “D*_1” of the stage “Day 1”, priority feature value types include types of feature value “A_1”, “D_1”, “B_1”, “E_1”, and “F_1” that are given places in an order of priority in this order, and, for the treatment decision model “D_2” of the stage “Day 2”, priority feature value types include types of feature value “C_2”, “D_2”, “B_1”, “A_1”, and “A_2” that are given places in an order of priority in this order. At this time, the priority feature value types of the stage “Day 2” include also the types of feature value “B_1” and “A_1” of “Day 1”. Note that places in an order of priority of types of feature value set for each treatment decision model may be set by any method.

Then, the treatment model generation unit 11 stores, on the model storage unit 17, the treatment decision models “D*_1”, “D*_2”, and “D*_3” for the stages generated as described above, and priority feature value types corresponding to each of the treatment decision models “D*_1”, “D*_2”, and “D*_3”. Note that the treatment model generation unit 11 may receive input of and acquire each treatment decision model of a stage prepared in advance, and priority feature value types corresponding to each of the treatment decision models, and store them on the model storage unit 17.

For each patient, the experience data generation unit 12 (collection unit) collects: first output f which is output, i.e. a treatment method, obtained when all types of feature value are input to each treatment decision model of a stage; and second output f which is output, i.e. a treatment method, obtained when some types of feature value in all the types are input to the treatment decision model of the stage, and outputs a determination result y as to whether or not the first output f and the second output f are identical. At this time, the experience data generation unit 12 varies the numbers and combinations of some types of all the types, and inputs sets of combined types of feature value to each treatment decision model. For example, regarding the treatment decision model “D*_1” of the stage “Day 1” illustrated in FIG. 5, combinations including increasing numbers of types are set starting from the one at the first place in the order of priority in the set priority feature value types, and combinations of types of feature value like (“A_1”), (“A_1”, “D_1”), (“A_1”, “D_1”, “B_1”), (“A_1”, “D_1”, “B_1”, “E_1”), . . . are input. At this time, the numbers of types to be combined are equal to or greater than one, and are smaller than the number of all the types (smaller than the predetermined number). Note that types of feature value not included in the combinations are input as “0”. Then, it is determined whether the second output f of respective sets of combined types of feature value, and the first output f of all the types of feature value are identical, and in the case where f=f′, the determination result y=1. In this manner, the experience data generation unit 12 generates experience data (aggregated data) including (X′: a set of combined types of feature value, y′: a set of determination results). Here, an example of the experience data (X′, y′) is illustrated below.

```
“In Case of Stage Day 1”
  (“A_1”, “0”, “0”, “0”, ..., 0)
  (“A_1”, “D_1”, “0”, “0”, ..., 0)
  (“A_1”, “D_1”, “B_1”, “0”, ..., 1)
  (“A_1”, “D_1”, “B_1”, “E_1”, ..., 1)
“In Case of Stage Day 2”
  (“C_2”, “0”, “0”, “0”, ..., 0)
  (“C_2”, “D_2”, “0”, “0”, ..., 0)
  (“C_2”, “D_2”, “B_1”, “0”, ..., 1)
  (“C_2”, “D_2”, “B_1”, “A_1”, ..., 1)
```

In the manner described above, the experience data generation unit 12 generates the experience data (X′, y′) for each stage by using patient information about each patient. Note that whereas the experience data generation unit 12 generates sets of combined types of feature value to be input to each treatment decision model of each stage by increasing the types in order of places in an order of priority, and varying the numbers and combinations of the types in the illustrated case in the description above, sets of combined types of feature value may be generated by randomly varying the numbers of combined types or randomly varying combinations of types.

For each stage, the feature value type setting unit 13 learns the relationship of the experience data (X′, y′), and generates a binary determination model H (second model) for determining the value (0 or 1) of y obtained with a set of combined types of feature value. Thereby, by receiving input of each set of combined types of feature value, the binary determination model H outputs an indication as to whether or not a treatment method (second output) which is output in the case where the set of combined types of feature value is input to a treatment decision model is identical to a treatment method (first output) which is output in the case where all the types of feature value are input to the treatment decision model. That is, with a set of combined types of feature value with which the binary determination model H outputs y=1, a treatment decision model outputs a treatment method which is identical to a treatment method decided from all the types of feature value, and accordingly it can be determined that a treatment may be decided with the set of combined types of feature value.

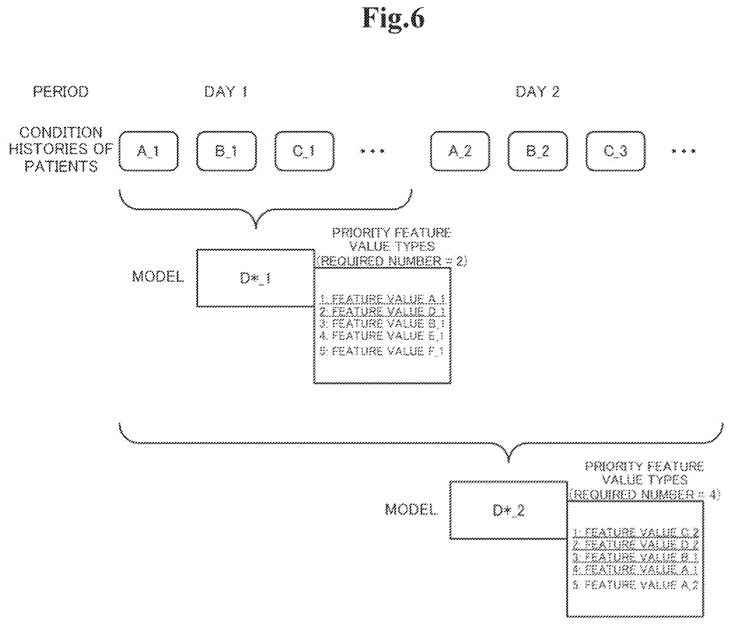

The feature value type setting unit 13 calculates a required number of types of feature value in priority feature value types set for a treatment decision model of each stage by using the binary determination model H generated for the relevant stage, by the way of thinking described above. Specifically, for each patient, the feature value type setting unit 13 inputs each varied set of combined types of feature value in patient information of each stage to the binary determination model H of the relevant stage, and checks the number of types of feature value with which output of y=1 is obtained. Then, for each stage, the average of the numbers of types with which output of y=1 is obtained regarding all the patients is calculated, and the average is treated as the required number of priority feature value types. By the processes described above, as an example, as illustrated in FIG. 6, the required number of priority feature value types set for the treatment decision model of the stage “Day 1” is calculated as “2”, and the required number of priority feature value types set for the treatment decision model of the stage “Day 2” is calculated as “4”. Note that the feature value type setting unit 13 may determine, for each patient, the minimum number of numbers of types of feature value with which output of y=1 is obtained, and may treat the average of the minimum numbers of all the patients as the required number. However, methods of calculating required numbers are not limited to the method described above, but required numbers may be calculated by any method such as a method in which the mode of numbers of types with which output of y=1 is obtained is treated as a required number.

The feature value type setting unit 13 further resets respective priority feature value types on the basis of the required number of priority feature value types set for a treatment decision model of each stage as described above. In particular, the feature value type setting unit 13 sets, as priority feature value types set for a treatment decision model of an earlier stage in temporally-consecutive stages, some of priority feature value types set for a treatment decision model of a latter stage.

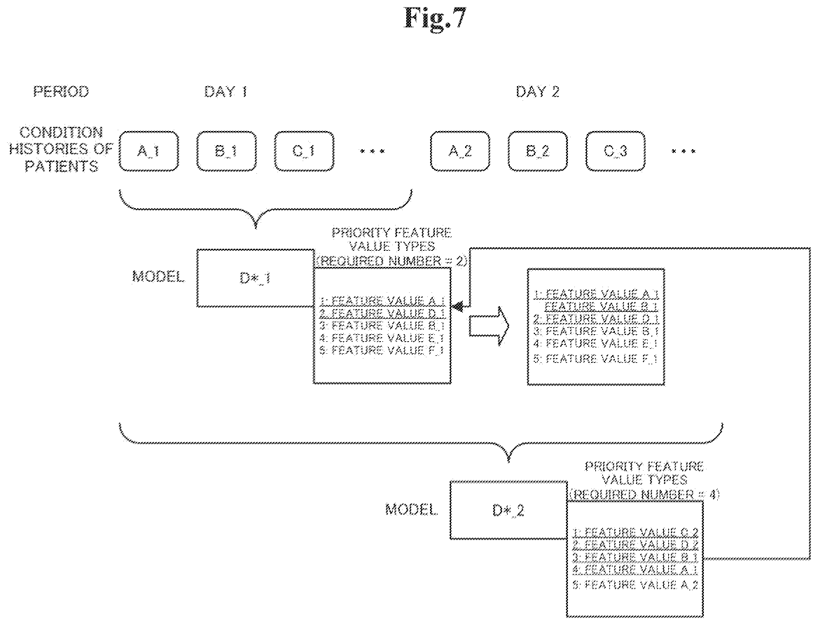

Here, a specific process of the feature value type setting unit 13 is described by taking, as an example, priority feature value types set for the treatment decision model of the stage “Day 1”, and priority feature value types set for the treatment decision model of the stage “Day 2” illustrated in FIG. 7. It is assumed that, at this time, the required number of priority feature value types of the stage “Day 1” is “2”, and the required number of priority feature value types of the stage “Day 2” is “4”. In this case, the feature value type setting unit 13 first checks the priority feature value types of “Day 2”, which is the latter stage, and extracts types of feature value representing a condition history of the patient of “Day 1”, which is the earlier stage, in the range of priority feature value types from the one at the first place in the order of priority to the one at the fourth place in the order of priority, which corresponds to the required number “4”. Then, the feature value type setting unit 13 extracts “B_1” and “A_1”, which are types of feature value at the third and fourth places in the order of priority of “Day 2”, which is the latter stage.

Next, the feature value type setting unit 13 checks the priority feature value types of the “Day 1”, which is the earlier stage, and checks whether priority feature value types in the range from the one at the first place in the order of priority to the one at the second place in the order of priority, which corresponds to the required number “2”, include the types of feature value “B_1” and “A_1” extracted from the stage “Day 2”. Then, since there is not the type of feature value

“B_1” in the range to the second priority feature value type of “Day 1”, which is the earlier stage, the type of feature value “B_1” is inserted and set in the priority feature value types of “Day 1”. That is, “B_1” is set as a type of feature value that should be acquired preferentially at the stage “Day 1”. At this time, as represented by an arrow in FIG. 7, the feature value type setting unit 13 inserts and sets the extracted type of feature value “B_1” at the intermediate position between the first priority feature value type and the second priority feature value type, which corresponds to the required number “2”, of “Day 1”, i.e. between the first priority feature value type and the second priority feature value type.

Note that methods by which the feature value type setting unit 13 extracts, from a latter stage, types of feature value to be inserted and set for an earlier stage are not limited to the method described above. For example, the feature value type setting unit 13 may extract types of feature value from some types of feature value used as input in the case where the determination result y=1 was obtained with experience data when the experience data was generated as described above. Further, at that time, a position in priority feature value types of another stage where an extracted type of feature value is inserted may be any position, and, for example, may be inserted at the beginning or end of the range from the first priority feature value type to the priority feature value type at a place corresponding to the required number or inserted at a position other than the position of the priority feature value type at a place corresponding to the required number. Further, priority feature value types of each stage do not necessarily have to be given places in an order of priority, and also required numbers described above do not have to be calculated. Types extracted on the basis of experience data as described above from priority feature value types of a certain stage may be inserted in priority feature value types of another stage. Furthermore, priority feature value types of each stage do not have to be set initially, and types of feature value extracted on the basis of experience data as described above may be newly set as priority feature value types.

### [Operation]

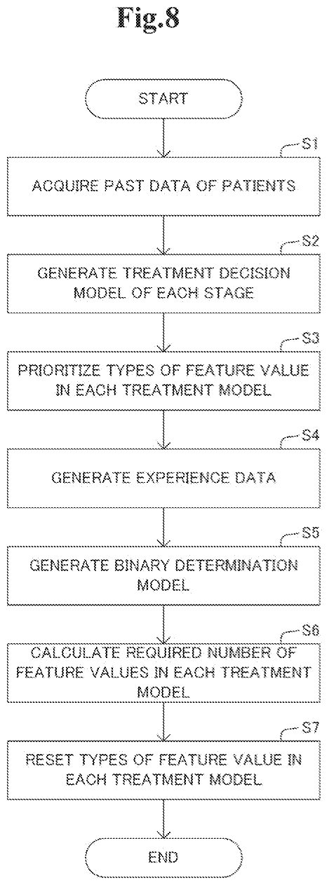

Next, operation of the information processing device 10 as described above will be described with reference to the flowchart of FIG. 8. The information processing device 10 first acquires patient information as learning data from the data management device 20 (step S1). As illustrated in FIG. 2, the patient information as the learning data includes, for each elapsed period, a plurality of types of feature value (“A_1”, etc.) representing condition histories of patients, and treatment histories (“Treatment a”, etc.).

Next, by using the patient information described above, the information processing device 10 generates the treatment decision models (“D*_1”, “D*_2”, etc.) that output treatment methods in accordance with input of condition histories of the patients for each stage which is an elapsed period like “Day 1” or “Day 2” as illustrated in FIG. 3 (step S2). At this time, as illustrated in FIG. 5, the information processing device 10 further sets, for each treatment decision model of a stage, priority feature value types which are types of feature value that are prioritized on the basis of the influence each of the types of feature value has on the treatment decision model (step S3).

Next, for each patient, by using the patient information described above, the information processing device 10 collects: the first output f which is output, i.e. a treatment method, obtained when all types of feature value are input to each treatment decision model of a stage; and the second output f which is output, i.e. a treatment method, obtained when some types of feature value in all the types are input to the treatment decision model of the stage, and also collects the determination result y as to whether or not the first output f and the second output f are identical. At this time, the information processing device 10 varies the numbers and combinations of some types of all the types, and inputs sets of combined types of feature value to each treatment decision model. Thereby, the information processing device 10 generates experience data including (X′: a set of combined types of feature value, y′: a set of determination results) (step S4).

Next, for each stage, the information processing device 10 learns the relationship of the experience data (X′, y′), and generates the binary determination model H for determining the value (0 or 1) of the determination result y obtained with a set of combined types of feature value (step S5). Then, the information processing device 10 calculates a required number of types of feature value in priority feature value types set for a treatment decision model of each stage by using the binary determination model H generated for the relevant stage (step S6). For example, for each patient, the information processing device 10 inputs each varied set of combined types of feature value in patient information of each stage to the binary determination model H of the relevant stage, checks the number of types of feature value with which output of y=1 is obtained, and thereby calculates a required number of priority feature value types.

Next, the information processing device 10 resets respective priority feature value types on the basis of the required number of priority feature value types set for a treatment decision model of each stage (step S7). For example, as illustrated in FIG. 7, the information processing device 10 sets, as priority feature value types set for a treatment decision model of “Day 1”, which is an earlier stage in temporally-consecutive stages, some of priority feature value types set for a treatment decision model of “Day 2”, which is a latter stage. Note that the set priority feature value types may be presented to a user who is a health care worker or the like by being output to a user terminal which is not illustrated. Thereby, the user can check the priority feature value types, and make decisions regarding correction.

As described above, the information processing device 10 according to the present disclosure first generates, for each treatment decision model of an elapsed period, experience data including determination results representing whether or not a treatment method output when all the types of feature value representing the condition of a human are input, and treatment methods output when sets of some types of feature value in all the types are input are identical. Then, on the basis of the experience data and for each treatment decision model of an elapsed period, it is possible to set necessary types of feature value prioritized for the model, and enhance the efficiency of collection of information about the necessary types of feature value.

In particular, prioritized and necessary types of feature value in a temporally-latter elapsed period can be set also as prioritized and necessary types of feature value in an earlier elapsed period, and the types of feature value can be acquired surely in the earlier elapsed period. Thereby, even when a smaller number of feature values than all types of feature values are input, it is possible to receive a proposal of a treatment method at precision similar to the precision at the time when all the types of feature value are input. As a result, it is possible to obtain a highly precise treatment method even with a smaller number of types of feature value, it is possible to suppress costs and time for collection of patient information, and it becomes possible to propose appropriate treatment methods promptly and easily. The information processing device 10 can assist decision-making by a health care worker such as a doctor, for example, in this manner.

### Second Exemplary Embodiment

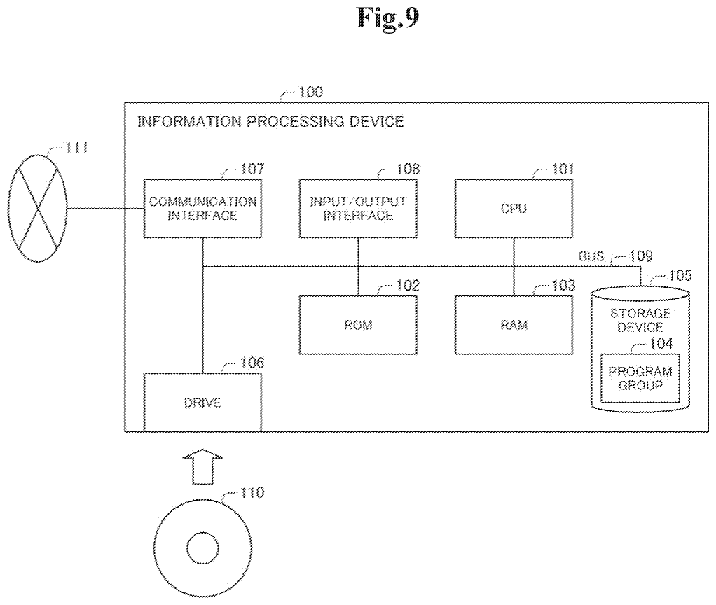

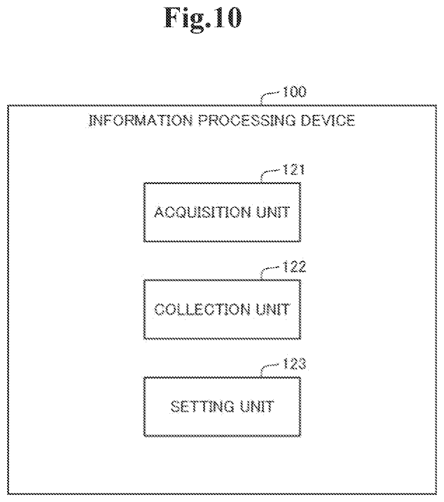

Next, a second exemplary embodiment of the present disclosure will be described with reference to FIGS. 9 to 10. FIGS. 9 and 10 are block diagrams illustrating a configuration of an information processing device according to the second exemplary embodiment. Note that the present embodiment illustrates the outline of the configuration of the information processing device described in the embodiment described above.

First, a hardware configuration of an information processing device 100 in the present embodiment will be described with reference to FIG. 9. The information processing device 100 includes a typical information processing device, having hardware configured as described below as an example.
Central Processing Unit (CPU) 101 (arithmetic unit)
Read Only Memory (ROM) 102 (storage device)
Random Access Memory (RAM) 103 (storage device)
Program group 104 to be loaded to the RAM 103
Storage device 105 storing thereon the program group 104
Drive 106 that performs reading and writing on a storage medium 110 outside the information processing device
Communication interface 107 connecting to a communication network 111 outside the information processing device
Input/output interface 108 for performing input/output of data
Bus 109 connecting the constituent elements

Note that FIG. 9 illustrates an example of the hardware configuration of the information processing device that is the information processing device 100. The hardware configuration of the information processing device is not limited to that described above. For example, the information processing device may include part of the configuration described above, such as without the drive 106. Further, the information processing device can use, instead of the CPU described above, a GPU (Graphic Processing Unit), a DSP (Digital Signal Processor), an MPU (Micro Processing Unit), an FPU (Floating point number Processing Unit), a PPU (Physics Processing Unit), a TPU (Tensor Processing Unit), a quantum processor or a microcontroller, a combination of these or the like.

Then, the information processing device 100 can construct and be equipped with an acquisition unit 121, a collection unit 122, and a setting unit 123 illustrated in FIG. 10 through acquisition of the program group 104 and execution thereof by the CPU 101. Note that the program group 104 is stored on, for example, the storage device 105 or the ROM 102 in advance, is loaded to the RAM 103 by the CPU 101, and is executed by the CPU 101 as needed. Further, the program group 104 may be supplied to the CPU 101 via the communication network 111, or may be stored on the storage medium 110 in advance and read out by the drive 106 and supplied to the CPU 101. However, the acquisition unit 121, the collection unit 122, and the setting unit 123 described above may be constructed by electronic circuits dedicated for realizing the means.

The acquisition unit 121 described above acquires models, each of which is generated for an elapsed period, and has learn to output measures for a human by receiving input of a plurality of types of feature value representing the condition of the human.

The collection unit 122 described above collects the first output that is obtained when a predetermined number of types of feature value are input to the model of each elapsed period, and the second output that is obtained when some types of feature value in the predetermined number of types of feature value are input to the model of the elapsed period.

The setting unit 123 described above sets types to be associated with the model of each elapsed period on the basis of the first output and the second output.

By being configured in the manner above, the present disclosure makes it possible to set types of feature value necessary for each model of an elapsed period. Thereby, it is possible to enhance the efficiency of collection of information about humans for measure proposals, and even when a smaller number of feature values are input, it is possible to propose highly precise measures corresponding appropriately to the conditions of the humans. Further, it is possible to suppress costs and time for collection of a smaller number of types of feature value, and it becomes possible to propose appropriate measures promptly and easily.

Note that the program described above can be supplied to a computer by being stored on a non-transitory computer readable medium of any type. Non-transitory computer readable media include tangible recording media of various types. Examples of non-transitory computer readable media include a magnetic recording medium (for example, flexible disk, magnetic tape, hard disk drive), a magneto-optical recording medium (for example, magneto-optical disk), a CD-ROM (Read Only Memory), a CD-R, a CD-R/W, and a semiconductor memory (for example, mask ROM, PROM (Programmable ROM), EPROM (Erasable PROM), flash ROM, and RAM (Random Access Memory)). Further, the program may also be supplied to a computer by being stored on a transitory computer readable medium of any type. Examples of transitory computer readable media include electric signals, optical signals, and electromagnetic waves. A transitory computer readable medium can supply programs to a computer via a wired communication channel such as an electric wire and an optical fiber, or a wireless communication channel.

While the present disclosure has been described thus far with reference to the exemplary embodiments and the like described above, the present disclosure is not limited to the embodiments described above. The configuration and details of the present disclosure can be changed within the scope of the present disclosure in various manners that can be understood by those skilled in the art. Further, at least one or more functions of the functions of the acquisition unit 121, collection unit 122 and setting unit 123 described above may be executed by an information processing device provided and connected at any location on a network, that is, may be executed by so-called cloud computing.

### SUPPLEMENTARY NOTES

The whole or part of the exemplary embodiments described above can be described also as the following supplementary notes. Hereinafter, outlines of the configurations of an information processing device, an information processing method, and a program according to the present disclosure will be described. However, the present disclosure is not limited to the following configurations.

### (Supplementary Note 1)

An information processing device comprising:
an acquisition unit that acquires a model that is generated for each elapsed period, and has learned by machine learning to output a measure for a human by receiving input of a plurality of types of feature value representing a condition of the human;
a collection unit that collects first output that is obtained when a predetermined number of types of feature value are input to the model of each elapsed period, and second output that is obtained when some types of feature value in the predetermined number of types of feature value are input to the model of each elapsed period; and
a setting unit that sets, on a basis of the first output and the second output, types to be associated with the model of each elapsed period.

### (Supplementary Note 2)

The information processing device according to supplementary note 1, wherein the collection unit collects the second output that is obtained when each varied set of some types of feature value in the predetermined number of types of feature value is input to the model of each elapsed period.

### (Supplementary Note 3)

The information processing device according to supplementary note 2, wherein the collection unit collects the second output that is obtained when each set of a varied number and/or combination of some types of feature value in the predetermined number of types of feature value is input to the model of each elapsed period.

### (Supplementary Note 4)

The information processing device according to supplementary note 2 or 3, wherein
the collection unit collects aggregated data including an indication as to whether or not the first output that is obtained from a model corresponding to an elapsed period is identical to the second output that is obtained when each varied set of some types of feature value in the predetermined number of types of feature value is input to the same model corresponding to the elapsed period, and
the setting unit sets, on a basis of the aggregated data, types to be associated with the model of each elapsed period.

### (Supplementary Note 5)

The information processing device according to supplementary note 4, wherein the setting unit sets, on a basis of the aggregated data, a required number of types to be associated with the model of each elapsed period, and sets the types on a basis of the required number.

### (Supplementary Note 6)

The information processing device according to supplementary note 5, wherein
types that are given places in an order of priority in advance are set in association with the model of each elapsed period, and
the setting unit resets the types on a basis of the places in the order of priority and the required number of the types associated with the model of each elapsed period.

### (Supplementary Note 7)

The information processing device according to supplementary note 6, wherein, on a basis of the places in the order of priority and the required number set for the types associated with a model of an earlier elapsed period in temporally-consecutive elapsed periods, and the places in the order of priority and the required number set for the types associated with a model of a latter elapsed period, the setting unit resets the types to be associated with the model of the earlier elapsed period.

### (Supplementary Note 8)

The information processing device according to any of supplementary notes 5 to 7, wherein the setting unit generates, for each elapsed period and on a basis of the aggregated data, a second model that receives input of each varied set of some types of feature value in the predetermined number of types of feature value, and outputs an indication as to whether or not the first output and the second output are identical, and sets the required number on a basis of output that is obtained when each varied set of some types of feature value in the predetermined number of types of feature value is input to the second model.

### (Supplementary Note 9)

An information processing method comprising:
acquiring a model that is generated for each elapsed period, and has learned by machine learning to output a measure for a human by receiving input of a plurality of types of feature value representing a condition of the human;
collecting first output that is obtained when a predetermined number of types of feature value are input to the model of each elapsed period, and second output that is obtained when some types of feature value in the predetermined number of types of feature value are input to the model of each elapsed period; and
on a basis of the first output and the second output, setting types to be associated with the model of each elapsed period.

### (Supplementary Note 10)

The information processing method according to supplementary note 9, further comprising collecting the second output that is obtained when each varied set of some types of feature value in the predetermined number of types of feature value is input to the model of each elapsed period.

### (Supplementary Note 11)

The information processing method according to supplementary note 10, further comprising:
collecting aggregated data including an indication as to whether or not the first output that is obtained from a model of an elapsed period is identical to the second output that is obtained when each varied set of some types of feature value in the predetermined number of types of feature value is input to the same model corresponding to the elapsed period; and
setting, on a basis of the aggregated data, types to be associated with the model of each elapsed period.

### (Supplementary Note 12)

The information processing method according to supplementary note 11, further comprising setting, on a basis of the aggregated data, a required number of types to be associated with the model of each elapsed period, and setting the types on a basis of the required number.

### (Supplementary Note 13)

The information processing method according to supplementary note 12, further comprising:
in association with the model of each elapsed period, setting types that are given places in an order of priority in advance, and
resetting the types on a basis of the places in the order of priority and the required number of the types associated with the model of each elapsed period.

### (Supplementary Note 14)

The information processing method according to supplementary note 13, further comprising, on a basis of the places in the order of priority and the required number set for the types associated with a model of an earlier elapsed period in temporally-consecutive elapsed periods, and the places in the order of priority and the required number set for the types associated with a model of a latter elapsed period, resetting the types to be associated with the model of the earlier elapsed period.

### (Supplementary Note 15)

The information processing method according to any of supplementary notes 12 to 14, further comprising generating, for each elapsed period and on a basis of the aggregated data, a second model that receives input of each varied set of some types of feature value in the predetermined number of types of feature value, and outputs an indication as to whether or not the first output and the second output are identical, and setting the required number on a basis of output that is obtained when each varied set of some types of feature value in the predetermined number of types of feature value is input to the second model.

### (Supplementary Note 16)

A computer readable storage medium storing thereon a program for causing a computer to execute processing to:
acquire a model that is generated for each elapsed period, and has learned by machine learning to output a measure for a human by receiving input of a plurality of types of feature value representing a condition of the human;
collect first output that is obtained when a predetermined number of types of feature value are input to the model of each elapsed period, and second output that is obtained when some types of feature value in the predetermined number of types of feature value are input to the model of each elapsed period; and
on a basis of the first output and the second output, set types to be associated with the model of each elapsed period.

### REFERENCE SIGNS LIST

10 information processing device
11 treatment model generation unit
12 experience data generation unit
13 feature value type setting unit
16 data storage unit
17 model storage unit
100 information processing device
101 CPU
102 ROM
103 RAM
104 program group
105 storage device
106 drive
107 communication interface
108 input/output interface
109 bus
110 storage medium
111 communication network
121 acquisition unit
122 collection unit
123 setting unit

1. An information processing device comprising:
at least one memory configured to store instructions; and
at least one processor configured to execute instructions to:
acquire a model that is generated for each elapsed period, and has learned by machine learning to output a measure for a human by receiving input of a plurality of types of feature value representing a condition of the human;
collect aggregated data including an indication as to whether or not first output is identical to each second outputs, the first output being obtained when a predetermined number of types of feature value are input to the model of each elapsed period, the second outputs being obtained when each varied set of some types of feature value in the predetermined number of types of feature value are input to the model of each elapsed period;
set, on a basis of the aggregated data, types of feature value to be associated with the model of each elapsed period; and
receive the set types of feature value modified by user using the terminal device.

2. The information processing device according to claim 1, wherein the at least one processor is configured to execute the instructions to collect the second outputs that are obtained when each set of a varied number and/or combination of some types of feature value in the predetermined number of types of feature value is input to the model of each elapsed period.

3. The information processing device according to claim 1, wherein the at least one processor is configured to execute the instructions to set, on a basis of the aggregated data, a required number of types to be associated with the model of each elapsed period, and set the types on a basis of the required number.

4. The information processing device according to claim 3, wherein
types that are given places in an order of priority in advance are set in association with the model of each elapsed period, and
the at least one processor is configured to execute the instructions to reset the types on a basis of the places in the order of priority and the required number of the types associated with the model of each elapsed period.

5. The information processing device according to claim 4, wherein the at least one processor is configured to execute the instructions to reset, on a basis of the places in the order of priority and the required number set for the types associated with a model of an earlier elapsed period in temporally-consecutive elapsed periods, and the places in the order of priority and the required number set for the types associated with a model of a latter elapsed period, the types to be associated with the model of the earlier elapsed period.

6. The information processing device according to claim 3, wherein the at least one processor is configured to execute the instructions to generate, for each elapsed period and on a basis of the aggregated data, a second model that receives input of each varied set of some types of feature value in the predetermined number of types of feature value, and outputs an indication as to whether or not the first output and the second outputs are identical, and set the required number on a basis of output that is obtained when each varied set of some types of feature value in the predetermined number of types of feature value is input to the second model.

7. An information processing method comprising:
acquiring a model that is generated for each elapsed period, and has learned by machine learning to output a measure for a human by receiving input of a plurality of types of feature value representing a condition of the human;
collecting aggregated data including an indication as to whether or not first output is identical to each second outputs, the first output being obtained when a predetermined number of types of feature value are input to the model of each elapsed period, the second outputs being obtained when each varied set of some types of feature value in the predetermined number of types of feature value are input to the model of each elapsed period;
on a basis of the aggregated data, setting types of feature value to be associated with the model of each elapsed period; and
receiving the set types of feature value modified by user using the terminal device.

8. The information processing method according to claim 7, further comprising setting, on a basis of the aggregated data, a required number of types to be associated with the model of each elapsed period, and setting the types on a basis of the required number.

9. The information processing method according to claim 8, further comprising:
in association with the model of each elapsed period, setting types that are given places in an order of priority in advance, and
resetting the types on a basis of the places in the order of priority and the required number of the types associated with the model of each elapsed period.

10. The information processing method according to claim 9, further comprising, on a basis of the places in the order of priority and the required number set for the types associated with a model of an earlier elapsed period in temporally-consecutive elapsed periods, and the places in the order of priority and the required number set for the types associated with a model of a latter elapsed period, resetting the types to be associated with the model of the earlier elapsed period.

11. The information processing method according to claim 8, further comprising generating, for each elapsed period and on a basis of the aggregated data, a second model that receives input of each varied set of some types of feature value in the predetermined number of types of feature value, and outputs an indication as to whether or not the first output and the second output are identical, and setting the required number on a basis of output that is obtained when each varied set of some types of feature value in the predetermined number of types of feature value is input to the second model.

12. A computer readable storage medium storing thereon a program for causing a computer to execute processing to:
acquire a model that is generated for each elapsed period, and has learned by machine learning to output a measure for a human by receiving input of a plurality of types of feature value representing a condition of the human;
collect aggregated data including an indication as to whether or not first output is identical to each second outputs, the first output being obtained when a predetermined number of types of feature value are input to the model of each elapsed period, the second outputs being obtained when each varied set of some types of feature value in the predetermined number of types of feature value are input to the model of each elapsed period;
on a basis of the aggregated data, set types of feature value to be associated with the model of each elapsed period; and
receive the set types of feature value modified by user using the terminal device.
